(19) United States
(12) Patent Application Publication
(10) Pub. No.: US 2017/0128187 A1
(43) Pub. Date: May 11, 2017

(54) ANTI-INTUSSUSCEPTION ILEAL STENT AND ITS USE AS AN ANTI-HYPERGLYCEMIC METHOD

(71) Applicant: Dr. Behrad Ziapour, Tabriz (IR)

(72) Inventor: Behrad Ziapour, Tehran (IR)

(21) Appl. No.: 15/414,595

(22) Filed: Jan. 24, 2017

(51) Int. Cl. A61F 2/04; A61B 17/12; A61F 2/91

(52) U.S. Cl. CPC A61F 2/04; A61F 2/91; A61B 17/12145; A61F 2002/045; A61F 2230/0069; A61F 2250/0067; A61F 2230/0028; A61F 2250/0098; A61F 2230/0021; A61F 2230/0023; A61F 2230/0008; A61F 2230/0006; A61F 2210/0014

(57) ABSTRACT

Selected diabetic patients reveal significant glycemic control after bariatric surgical or endoscopic duodenal bypass. This controlling effect appears to occur partly because of an enhanced Glucagon-Like Peptide 1 (GLP1) release from intestinal L-cells. These procedures may also cause complications such as nutritional deficiencies. The present invention provides a stent. The method that places this stent in the ileum to activate the endogenous GLP1 release is a safer alternative to control the diabetes mellitus. However, the less mobile wall of the ileum next to the proximal stent rim can trigger an unwanted telescoping of the intestine, which is known as intussusception. An embodiment of this invention illustrates the characteristics of a stent that can prevent a likely intussusception and consequently be more confidently applied in the ileum to maintain or restore the ileal patency in general or for its anti-hyperglycemic effect in particular.

### BACKGROUND OF THE METHOD AND INVENTION

The treatment and control of the diabetes mellitus are worldwide concerns. The demonstrated success of Glucagon-like Peptide 1 (GLP-1) to lower glycemia has led to the approval of the medications to treat patients with type-2 diabetes. GLP-1 and Gastrin Releasing Peptide (GRP) are the key peptides in the glycemic regulation and are released from L-cells in the ileum and myenteric postganglionic neurons, respectively. The enhancement of the release of GLP-1 from the ileum is one of the known mechanisms, by which some bariatric surgical or endoscopic methods can successfully control the plasma glucose levels in selected patients. This control is partly provided by diverting nutrients from the proximal region of the gastrointestinal (GI) tract and delivering these incompletely digested nutrients to the ileum. However, because of the gastrointestinal tract bypass, almost all such methods cause considerable adverse consequences on nutrients and drugs absorption. Even a reversible method such as GI liner is not recommended for long-term implantation. Until now, GLP-1 or GRP secretion enhancement has never been discussed or attempted through a direct ileal stimulus or ileal device. In this context, embodiments of the present invention are ileal stents, which introduce anti-hyperglycemic effects via direct ileal stimulus. Examples of the ileal implanted stents of the present invention, and the expected mechanism of their action neither distort the GI tract structure nor interrupt the GI absorptive surface. Thus, the nutrient deficiencies that commonly result from current bariatric interventions are not expected.

### SUMMARY OF THE INVENTION

According to an embodiment of the present invention, an ileal stent activates the enteric sensory path, which subsequently activates vagus efferent fibers. These efferent fibers stimulate L-cells directly via Acetylcholine (Ach) and indirectly via GRP to secret more endogenous anti-glycemic GLP-1. By activating the endogenous GLP-1 secretion, anti-hyperglycemic ileal stents can control the plasma glycemic levels of diabetic patients. This therapeutic method is expected to have fewer complications and be less invasive than the current bariatric operative or endoscopic interventions with similar mechanisms in diabetic patients. However, an ileal wall fixed by the stent can theoretically trigger an intussusception. Intussusception is the telescoping of one portion of the intestine (intussusceptum) into the lumen of the intestine immediately distal to it (the intussuscipiens). Abnormal longitudinal forces on the bowel wall of different causes and abnormal peristalsis are considered responsible for this phenomenon. The present invention has an anti-intussusception structure.

### BRIEF DESCRIPTION OF THE DRAWINGS

FIG. 1 shows the mechanism of the action of the present invention.

FIG. 2 shows a stent as one embodiment of the present invention.

FIG. 3 shows the longitudinal view of the stent.

FIG. 4 shows the elements of the anti-intussusception structure at the proximal end of the stent.

FIG. 5 shows how the collar and legs are connected to the body of the stent.

FIG. 6 shows a schematic of the intussusception pathogenesis.

### DETAILED DESCRIPTION OF THE INVENTION AND METHOD

This description exemplifies the core concept of the method and principles of the invention, but they are not limited to the particular embodiments illustrated herein.

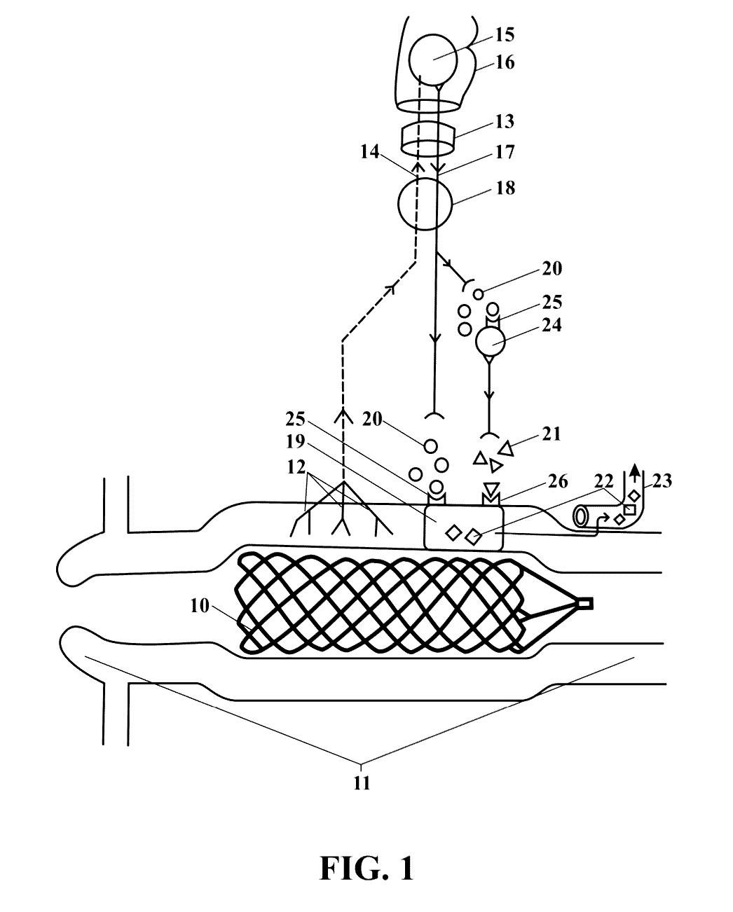

As shown in FIG. 1, an ileal stent (10) placed in the terminal ileum (11) is proposed to activate the enteral sensory branches (12), and this stimulus is transmitted via the vagus nerve (13) afferent fiber (14) toward the vagus nucleus (15) in the brain stem (16). This received stimulus subsequently activates the vagus efferent (17). After passing through the celiac ganglion (18), the ileal branches of this efferent path stimulate the ileal L-cells (19) either directly via acetyl-choline Ach (20) or indirectly via GRP (21) to enhance the GLP1 (22) secretion. GLP1 enters the microvasculature (23) of the intestine and systemically decreases glycemia. The GRP is a neuropeptide that is locally released from the postganglionic parasympathetic fibers of GRPergic neurons (24) localized to some myenteric ganglions. In fact, L-cells have Ach receptors (25) and GRP receptors (26), which make them sensitive to the stimulatory effects of both mentioned chemicals.

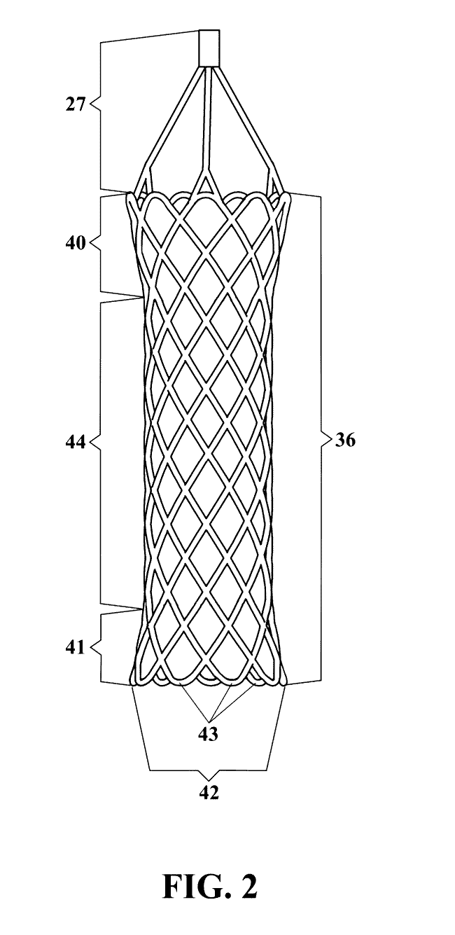

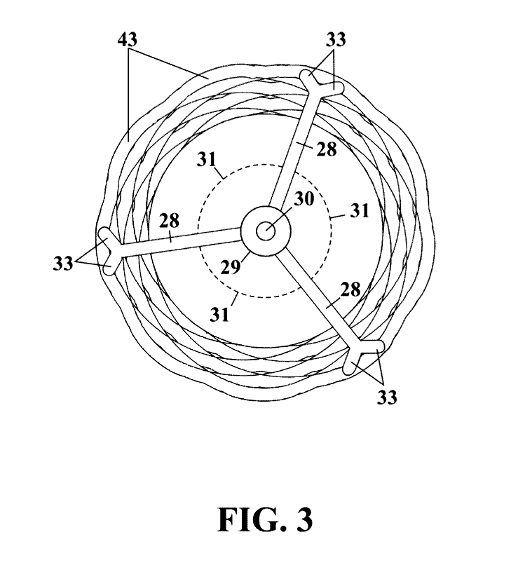

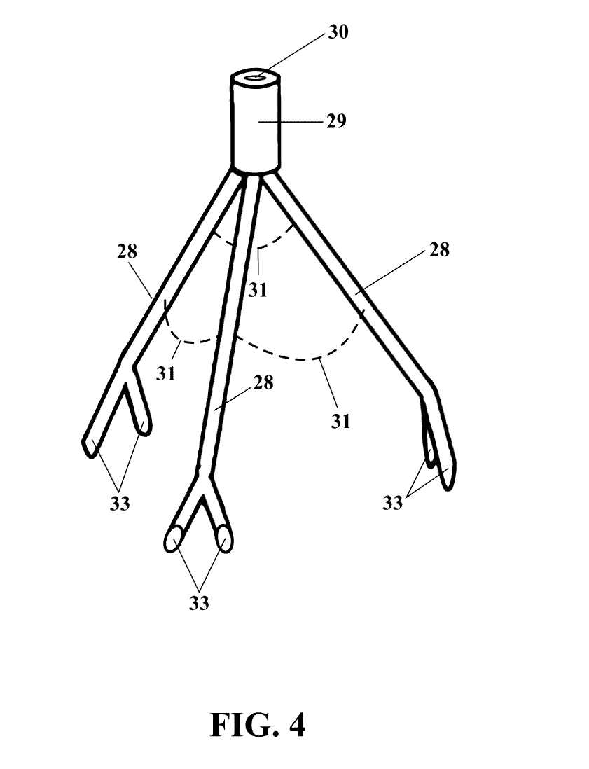

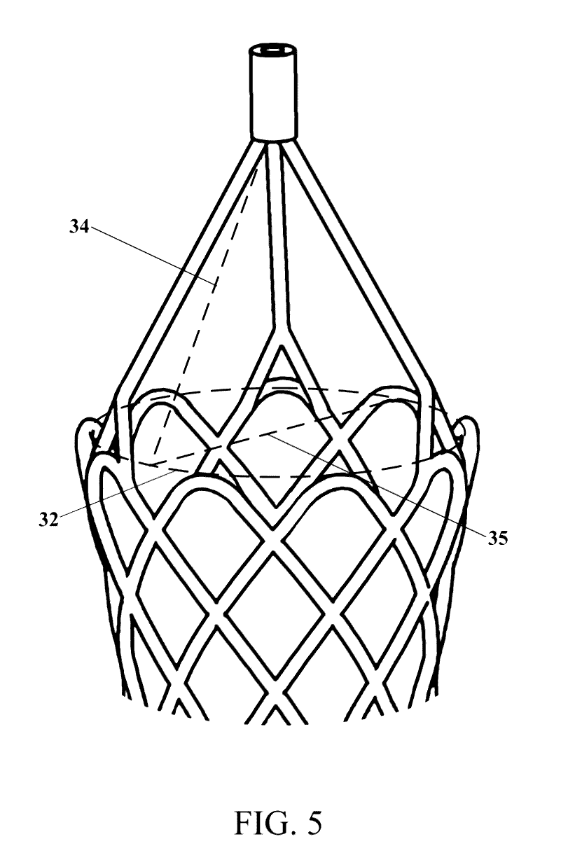

FIGS. 2-5 show a stent or part of a stent with a tripod structure (27) at its proximal end. A tripod, as used in the anti-intussusception ileal stent AIIS, refers to a structure with 3 equal legs or spokes (28) connected at their proximal ends to a central collar (29). This is still within the scope of the invention for the stent to have other structures (not shown in the figures, such as uni- or multi-spoke/leg) instead of a tripod-like structure (tri-spoke/leg). The central collar axis is superposed on the stent axis. The central collar allows the guide-wire of a delivery device to pass through its axial canal (30) as shown in FIGS. 3 and 4. In a preferred embodiment, from the central collar, three legs fan out to make three equal vertex angles (31) from one another, each of which is always <120 degrees. Each spoke/leg connects to the stent inlet (32) rim, as shown in FIG. 5, via a two-prong fork (33), as shown in FIGS. 3 and 4. In a preferred embodiment to provide a stronger stand, the prongs are lambda (λ)-like extensions of the stent inlet rim. The legs converge in the central collar with a moderate slope. However, it is within the scope of the invention for the tripod having legs with other shapes, for example: straight, curved, sinusoidal or a combination of these forms. The legs are resilient, and the compression on the outer edge of the legs hold them packaged in a straight or partially straight form in the delivery device, while a guide wire can pass through the axial canal of the central collar. When the delivery device tube is withdrawn at the intended site of placement, the legs of the tripod are biased away because of the aforementioned resilience.

As shown in FIG. 5, in a preferred embodiment, the tripod widest slant height (34) is equal or larger than the stent widest inlet diameter (35). This setup enables the tripod to pass the largest possible indigested food that may pass through a normal ileum diameter.

Although in one embodiment, the legs and feet of the anti-intussusception compartment of the AIIS are made of wires with circular cross section, the anti-intussusception elements can be made with other cross sections such as square or triangular ones.

The elements of the anti-intussusception tripod structure (27) can be attached or secured to each other and to the stent part (36) in any manner such as welding, soldering, brazing, wrapping, interweaving and any combination thereof.

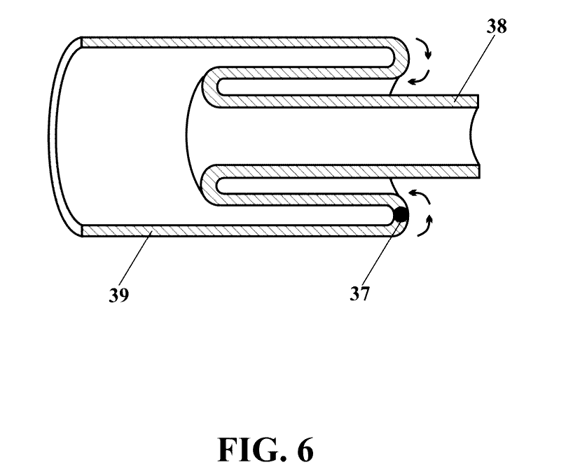

As shown in FIG. 6, an ileal wall, which is fixed by a lead point (37) that can be either a mural pathology or a stent, can theoretically trigger an intussusception. Intussusception is the telescoping of one portion of the intestine (intussusceptum) (38) into the lumen of the intestine immediately distal to it (the intussuscipiens) (39). Abnormal longitudinal forces on the bowel wall of different causes and abnormal peristalsis are considered responsible for this phenomenon, which is particularly more common in ileum than other intestinal segments. To prevent this possible complication, the anti-intussusception ileal stent has the proximal tripod in its structure, which makes it distinct from other stents. Intussusception telescoping is an integrated circumferential advancement, which indicates that telescoping is impossible when even a single point on the ileal wall circumference remains unfolded. Through unfolding any telescoping between the central collar and the stent inlet rim, the tripod legs prevent likely intussusceptions, which are triggered by the ileal free and fixed wall interface adjacent to the stent inlet rim.

The inventive anti-intussusception ileal stents comprise the embodied anti-intussusception structure (27), which is attached to a self-expandable stent part (36), wherein the aforementioned stent part comprises; a woven, knitted or laser-cut mesh cylinder or braided wire tubule that exerts self-expansive forces until it reaches a fixed maximum diameter. The stent part can also be uncovered or covered by a membrane. If covered, polyurethane, silicone, and expanded polytetrafluoroethylene are examples of covering materials. To locate the device during and after the insertion in the intestine, the present invention has radiopaque markers at different locations of the anti-intussusception structure and stent part.

In an embodiment, the stent part is hollow and cylindrical, but in a preferred embodiment as shown in FIGS. 2 and 5, the stent part is a hollow cylinder with flare-like ends (40)(41), an outlet (42), an inlet (32) and looped-end rims (43).

The stent part, anti-intussusception structure and delivery device can have different sizes. However, in at least one preferred embodiment and regarding the average diameter of ileum (2.5 cm) in adults, the unconstrained (expanded) outer diameter of proximal flare (40), body (44), distal flare (41) and unconstrained length of the said stent part are 26±1 mm, 20±1 mm, 24±1 mm and 75±5 mm, respectively. For the average colon length (150 cm), the delivery system length should be 160 mm in this later embodiment.

It is also within the scope of the invention for the stent part and anti-intussusception structure to have different radial forces, axial forces, flexibility, shortening ratio, radiopacity, and tractability. However, in the preferred embodiment of the present invention, the stent part has a radial force >4.00 N but lower than the force that can introduce a 30-35 mmhg pressure on the ileal wall. The lower limit decreases the migration risk, and the upper limit decreases the risk of mucosal ischemic injury.

The stent part (36) and anti-intussusception structure (27) can be made from biocompatible materials such as polymers, composites, biocompatible biodegradable materials, polycarbonate copolymers, stainless steel, titanium, platinum, tungsten, gold, alloys (Elgiloy, Phynox, MP35N alloy, nickel-titanium alloys (Nitinol)) or any combination of these materials.

In the preferred embodiment, the stent part of the present invention is made of a flexible and shape-memory material such as Nitinol, and the anti-intussusception structure is made of Nitinol or a material more resistant to the axial force and/or less flexible. These preferred stronger axial forces of anti-intussusception structure legs hold the legs against food passage and likely intussusceptions. Also, to maintain the conformability of the stent in the ileum, the axial force of the stent part should not exceed 4.00 N.

To prevent stent migration, one embodiment of the present invention has the stent part with anchoring flaps, quill filaments or struts in all or some parts of the stent and/or different levels of segmental radial forces over the entire length of the stent part.

In some embodiments, at least one portion of the AIIS is configured to include one or more therapeutic agents, which coat the bare mesh or covering membrane and are released at the stent implantation area or adjacent areas. Examples of the therapeutic agents are various anti-hyperglycemic agents.

In one preferred embodiment, the therapeutic agent has indigestible and non-absorbable short-chain fatty-acid-like branches. Short-chain fatty acids are direct stimulators of intestinal L-cells to secret GLP-1. Thus, an enhanced anti-hyperglycemic effect is expected when such Fat-Coated Stents (FCSs) are used.

In one embodiment, the AIIS is packaged in a compressed form and constrained on a delivery device. Then, ileoscopy through the stoma with a trans-endoscope helps place the stent over a wire via the delivery device. However, because of the anti-intussusception structure at the proximal end, delivery devices that are designed for the proximal stent release are preferred.

1. An anti-intussusception ileal stent comprising:
a) a generally cylindrical main body structure; and
b) an anti-intussusception structure capping one end of said cylindrical main body structure, wherein, when said stent is placed within the distal part of small intestine (ileum), said anti-intussusception structure prevents instances of intestinal invagination and intussusception by effectively blocking sliding of a proximal and adjacent part of the ileum into stent part of said ileum in a telescopic manner.

2. The anti-intussusception ileal stent of claim 1 in which said cylindrical body is of woven metallic wire material, woven, knitted or laser-cut mesh cylinder or braided wire tubule.

3. The anti-intussusception ileal stent of claim 1 in which said generally cylindrical main body structure exerts self-expansive forces until it reaches a fixed maximum diameter.

4. The anti-intussusception ileal stent of claim 2 in which said anti-intussusception structure comprises a collar and at least one spoke, said collar comprising an axially bored cylinder with a smaller diameter than the diameter of said cylindrical main body, in which said capping is implemented by said at least one spoke linking said collar to the cylindrical main body by circumferentially connecting to both said one end of the cylindrical main body structure and one end of said collar.

5. The anti-intussusception ileal stent of claim 1 in which said cylindrical main body and the anti-intussusception structure are made from material made from a material group comprising biocompatible materials, polymers, composites, biocompatible materials, biodegradable materials, polycarbonate copolymers, stainless steel, titanium, platinum, tungsten, gold, Elgiloy alloy, Phynox alloy, MP35N alloy, nickel-titanium alloys, Nitinol alloy and any combination thereof.

6. The anti-intussusception ileal stent of claim 5 in which said anti-intussusception structure comprises of a collar and at least one spoke, said collar comprising an axially bored cylinder with a smaller diameter than the diameter of said cylindrical main body, in which said capping is implemented by said at least one spoke linking said collar to the cylindrical main body by circumferentially connecting to both said one end of the cylindrical main body structure and one end of said collar.

7. The anti-intussusception ileal stent of claim 4, in which said at least one spoke further comprises a lambda (λ-) shape structure at its junction to the inlet rim of said one end of said cylindrical main body.

8. The anti-intussusception ileal stent of claim 7 further comprising a cover for the generally cylindrical main body structure, said cover being made from material from the group comprising polyurethane, silicone, and expanded polytetrafluoroethylene, said cover carrying radiopaque markers at least in some locations.

9. A tripod connected to one end of a substantially cylindrical stent, said tripod comprising;
Three legs with equal length, said legs fan out making equal angles to each other,
a central collar aligned with on the stent axis such that a delivery device guide wire passes through the central collar,
wherein compression on the outer edge of legs hold them packaged in a substantially unbent form within the delivery device while a guide wire passes through the axial canal of the central collar, and when the delivery device tube is withdrawn at an intended site of placement, legs of tripod are biased away by the resilience of the legs, and the tripod's widest slant height is equal to or larger than the stent's widest inlet diameter.

10. The tripod of claim 9, in which the shape of said legs comprises straight, curved, sinusoidal or a combination thereof.

11. The anti-intussusception ileal stent of claim 1 wherein;
elements of the anti-intussusception structure have circular, square, triangular, ellipse or hexagon cross sections,
said elements are attached to each other as well as to the stent body, and
said cylindrical main stent part structure has flare-like ends.

12. The anti-intussusception ileal stent of claim 1 wherein;
said cylindrical main stent has structures comprising anchoring flaps, quill filaments or struts in all or some parts of the stent.

13. The anti-intussusception ileal stent of claim 1 wherein the local pressure applied by the stent body on the ileal's wall is variable along the stent's length hence creating better grip.

14. The anti-intussusception ileal stent of claim 11 wherein at least a portion of the stent main body includes one or more therapeutic agents by coating the bare mesh or said covering membrane with said therapeutic agents, said therapeutic agents to be released to the stent implantation area or adjacent areas.

15. The therapeutic agent of claim 14 which is made from a material presenting indigestible and non-absorbable short chain fatty acid-like branches.

16. An anti-hyperglycemic method comprising of the following steps;
1. Operating an ileoscopy to introduce the stent delivery device,
2. Placing the anti-intussusception ileal stent within the ileum about 10 cm proximal to the ileocecal valve, such that the stent expands and applies a modest, longitudinally non-uniform radial force to the ileal wall, causing stretch of the ileal wall; subsequently, enteral sensory path is activated by said ileal stretch. vagus efferent of said enteral sensory path transmits sensory stimulation toward vagus central nucleus, vagus central nucleus then starts an efferent response, passing through Celiac ganglion, vagus efferent response arrives in ileal wall myenteric system, terminal branches of the said efferent fibers, release acetylcholine, said acetylcholine stimulates post-ganglionic parasympathetic GRPergic neuron, GRPergic neurons secret Gastrin Releasing Peptide GRP, Both GRP and acetylcholine stimulates the secretion of Glucagon like peptide 1 GLP-1 from ileal L-cells, and GLP-1 lowers and regulates the glycemia.
